(19) United States
(12) Patent Application Publication
(10) Pub. No.: US 2024/0238118 A1
(43) Pub. Date: Jul. 18, 2024

(54) Cooling Shirt for Workers in Hot Environments

(71) Applicant: TDA Research, Inc, Wheat Ridge, CO (US)

(72) Inventors: David P. Eisenberg, Denver, CO (US); Robert James Copeland, Wheat Ridge, CO (US); Sara Locke, Golden, CO (US); Alexander Riffe, Broomfield, CO (US); Girish Srinivas, Broomfield, CO (US)

(21) Appl. No.: 18/413,629

(22) Filed: Jan. 16, 2024

Related U.S. Application Data
(60) Provisional application No. 63/439,264, filed on Jan. 16, 2023.

(51) Int. Cl. A61F 7/02; A41D 13/002; A41D 13/005; A61F 7/00

(52) U.S. Cl. CPC A61F 7/02; A41D 13/0025; A41D 13/0053; A61F 2007/0018; A61F 2007/006; A61F 2007/0067; A61F 2007/0234; A61F 2007/026

(57) ABSTRACT

A cooling garment for use by workers in hot, humid climates comprising: a moisture-wicking under layer; an outer layer with an air permeability less than 200 L/m²/s when subjected to 125 Pascals of air pressure; at least one air channel formed between the layers and supported in at least some locations by a porous filler material; and an above ambient pressure gas supply attached operably to the inlet of the channel. Also a cooling garment comprising: a moisture-wicking under layer; an outer layer; one large air channel formed between the layers; tacks connecting the outer layer to the moisture-wicking under layer to prevent ballooning; and an above ambient pressure gas supply operably attached to the inlet of the channel.

### REFERENCE TO RELATED APPLICATIONS

This application claims the benefit of U.S. provisional application 63/439,264, filed Jan. 16, 2023 (titled Cooling Shirt for Workers in Hot Environments, by Girish Srinivas, David P Eisenberg, Sara Locke, and Robert James Copeland, attorney docket number 21-6) which is incorporated by reference herein in its entirety.

### RIGHTS TO INVENTION UNDER FEDERAL RESEARCH

This invention was made in part using U.S. government funding through the U.S. Navy contract #N00014-19-9-0015. The government has certain rights in this invention.

### BACKGROUND OF THE INVENTION

Fire fighters, first responders, military personnel, construction workers, and people in other labor-intensive occupations work in hot, humid, and dangerous environments where they risk overheating, which can lead to loss of effectiveness, heat stroke, and significant cognitive impairment. Couple these conditions with the heavy protective gear that many workers must wear, and they are left in an inescapable, hot, sweaty, and potentially dangerous state. Breaks which could prevent overheating are often skipped due to time pressures of their work, resulting in frequent cases of heat-related illnesses, some of which are life threatening. A 70-kilogram (154 lb), physically fit individual engaged in very heavy work (such as digging with a shovel) generates about 2000 Btu/hr (586 W) of heat. The heat capacity of tissue is about 3.5 kJ/kg° C. (0.836 Btu/lb° F.) and in the absence of any heat losses, absorbing this much heat will raise the body's core temperature from 37° C. (98.6° F.) to 40° C. (104° F.) in about 20 min. When the body's core reaches a temperature of 104° F., heat stroke occurs. This is a medical emergency where the body loses its ability to regulate temperature, and if untreated, can lead to permanent disability or death. At somewhat lower core body temperatures (before the body loses its thermoregulatory capabilities), heat exhaustion occurs and is also serious. Symptoms of heat exhaustion include: heavy sweating, paleness, muscle cramps, fatigue, weakness, dizziness, headache, nausea, and sometimes brief loss of consciousness (fainting). Even mild overheating can cause painful muscle cramps and heat rash.

Sweating is the body's way of naturally releasing excess heat. Once the body's internal temperature begins to rise, the vast network of sweat glands begin producing sweat to cool itself. The cooling of the human body, however, is not achieved through sweating itself but by the evaporation of the sweat. In dry climates, the body provides heat necessary for evaporation while the low ambient humidity lowers the driving force for the evaporation of the sweat, allowing for sweating that leads to cooling. In hot and humid conditions, this is not the case, and the body will not feel cool simply by sweating. Taking multiple work/rest cycles can prevent dangerous overheating in these cases, but the inherent pressures to complete work quickly in many occupations cause workers to decline necessary breaks. They must instead rely on air conditioning, ice, circulating liquid cooling systems, cold packs, and phase change materials to cool themselves. However, these methods are frequently inapplicable or impractical for workers due to their size, weight, requirements for re-freezing or re-cooling, and/or dangers of overcooling. There is a need for a cooling method that works in tandem with the body's rate of metabolic heat production, so the amount of cooling taking place is exactly what the body needs at a given point in time.

Layers of clothing and protective equipment provide yet another obstacle to cooling a worker by restricting air flow, trapping sweat on a person's body or in the layer of clothing nearest to the person's skin, and adding weight and heat to be dissipated. In hot, humid climates this can be extremely uncomfortable (distraction, leading to improper job completion) and potentially dangerous (leading to heat exhaustion and heat stroke). Heat stress when wearing clothing and protective gear and the effectiveness of existing personal cooling technologies (frequently referred to as microclimate technologies) has been studied to determine both the physiological and psychological effects of overheating on the ability of people to perform a wide variety of tasks that require different levels of exertion (Cadarette, B. S.; Cheuvront, S. N.; Kolka, M. A.; Stephenson, L. A.; Montain, S. J. and Sawka, M. N. (2006) “Intermittent Microclimate Cooling During Exercise Heat Stress in U.S. Army Chemical Protective Clothing,” Ergonomics, Vol. 49, No. 2, 209-219).

Overall, there lacks an effective cooling mechanism for workers in hot, humid climates. Many standard cooling methods are heavy, require re-freezing or refrigeration, only possess a single level of cooling while the body's heat production is variant, or leave the worker drenched in uncomfortable sweat. Workers need a lightweight and long-lasting wearable cooling device that can keep up with their body's metabolic heat generation even when inducing maximal effort in the most humid, hot climates. This will lower the risk for heat stroke and other heat-related health issues in workers in hot environments, allowing them to continue working comfortably at whatever rate they choose.

U.S. Pat. No. 9,635,889 attempts to address these problems. However, this teaching is dependent on an incorrect assumption of hydraulic diameter. By only wetting the bottom surface of a channel, hydraulic diameter is actually 4 times the thickness between the two layers, not the calculation relied on in U.S. Pat. No. 9,635,889. Thus, the garment teaches away from ideal channel dimensions, which results in decreased cooling effectiveness. In addition, it requires intense labor to form the many small, circular, open-air tubes as taught in U.S. Pat. No. 9,635,889, making mass production unrealistic. Manufacturing feasibility is necessary to lower the product's cost and promote implementation into the workplace. The tubes in U.S. Pat. No. 9,635,889 are susceptible to collapse under heavy protective layers, which leads to areas of failed air flow and decreased cooling of the wearer.

U.S. Pat. No. 8,756,718B2 and U.S. Pat. No. 9,772,166B2 teach garments for cooling underneath protective gear with spacers to hold the garments away from the user, eliminating risk of air flow channels collapsing. The garments in U.S. Pat. No. 8,756,718B2 and U.S. Pat. No. 9,772,166B2 rely on natural convection and sweating to provide cooling through the air channels, yet, as described above, this is not sufficient means for cooling in hot, humid climates. Further, U.S. Pat. No. 8,756,718B2 teaches away from mechanical mechanisms for inducing air flow.

US2005/0246826 teaches a garment for use beneath a bullet proof vest with raised ribs to form channels, and a fan. However, US2005/0246826 teaches the use of an exhaust fan which pulls air out of the channels rather than blowing air across them, and teaches away from fans that induce positive air pressure through the channels.

Limitations of the prior art are at least one of the following: (i) they teach away from the idealized measurements for promoting active airflow; (ii) they are susceptible to failure when worn underneath additional heavy layers of clothing; (iii) they are not effective in hot and humid climates where sweating cannot provide cooling; (iv) they require complex manufacturing, making wide implementation unrealistic; (v) they improperly target only certain areas of a wearer for cooling, leading to uneven and ineffective cooling.

For the foregoing reasons, there is a need for a lightweight cooling garment that is easy to manufacture and effectively cools a wearer of the garment underneath other layers of clothing, potentially heavy protective gear, and completing physical tasks in hot, humid climates.

### SUMMARY OF THE INVENTION

The present disclosure relates to cooling garments and solves the limitations of the prior art. The invention is a cooling garment, which can prevent heat related illnesses, especially heat exhaustion and heat stroke. A feature of this garment is that it relies on a simple construction that can be manufactured at a minimal cost. Another feature of this garment is that it does not require consumables (aside from batteries in a portable embodiment of the garment), water, or cumbersome equipment. Another feature of this garment is that it regulates the wearer's temperature to prevent both overheating and overcooling.

The present disclosure is a cooling garment, comprising: a moisture-wicking under layer (41); an outer layer attached to the outer surface of the moisture-wicking under layer (43), wherein the outer layer has an air permeability less than 200 L/m²/s when subjected to 125 Pascals of air pressure; at least one air channel (44) between the moisture-wicking under layer and the outer layer, wherein the air channel has a width of 0.25-4 inches, and wherein the air channel is supported in at least some locations by a porous filler material with a height of 2-10 millimeters; and an above ambient pressure gas supply (64) operably attached to an inlet to the channel (63) to thereby cool the wearer of the cooling garment, wherein the gas supply forces air through the channel at a total flow rate of 10 to 40 cubic feet per minute. In a preferred embodiment, the channel width is 0.5-2 inches, more preferably the channel width is 0.75-1.5 inches. The garment can have a variety of number of channels combined with varying channel heights and widths within the recommended ranges. In one embodiment, the garment has 30-50 air channels. In another embodiment, the garment has 30-50 air channels with a width of 1 inch and a height of 2 millimeters.

In an embodiment, the present disclosure has a moisture-wicking underlayer which is made from a fabric which has a water-wicking capability producing a water rise of 1-9 centimeters, measured at 60 seconds after exposing a bottom 5 millimeters of a swatch of the fabric into a beaker of water. In a more preferred embodiment, the water-wicking capability produces a water rise of 3-8 centimeters, and more preferably a water rise of 6-7.5 centimeters. In another embodiment, the moisture-wicking under layer has a permeability which changes from the inlet to an outlet to encourage air flow across a wearer's torso or body part covered by the garment. In a preferred embodiment, the permeability of the moisture-wicking under layer is 100-10,000 L/m²/s. In a more preferred embodiment, the permeability is 250-5,000 L/m²/s, and most preferably 500-2,000 L/m²/s. In another preferred embodiment, the garment has a total flow rate of 25 ft³ per minute. In a further embodiment, the above ambient air pressure gas supply is either a compressed air tank, a blower, or a battery-powered fan.

The present disclosure also relates to a cooling shirt, comprising: a moisture-wicking under layer (92); an outer layer attached to the outer surface of the moisture-wicking under layer (93), wherein the outer layer has an air permeability less than 200 L/m²/s when subjected to 125 Pascals of air pressure; one large air channel between the moisture-wicking under layer and the outer layer (94), wherein the air channel is supported by a porous filler material with a height of 2-10 millimeters; an array of tacks (82), wherein the tacks connect the moisture-wicking under layer to the outer layer to prevent ballooning (91); and an above ambient pressure gas supply operably (81) attached to an inlet to the channel to thereby cool the wearer of the cooling garment, wherein the gas supply forces air through the channel at a total flow rate of 10 to 40 cubic feet per minute.

The tacks can be arranged in a variety of distances from one another. Preferably, the tacks are 0.25-4 inches apart. In an embodiment, the tacks are spaced 2 inches apart. In another embodiment the tacks are spaced 1 inch apart.

### BRIEF DESCRIPTION OF DRAWINGS

FIG. 1. Schematic of one method of attaching air flow channels to the moisture-wicking garment.

FIG. 2. Schematic of air channels with an example of flow direction from fan.

FIG. 3. Schematic of cooling garment in use.

FIG. 4. Diagram of how air channels are formed between layers of the garment. Cross-sectional view of channels (a) with porous filler material support (b) without any filler material.

FIG. 5. Schematic of one method of attaching porous filler material only in some sections of the cooling garment.

FIG. 6. Schematic of the cooling garment showing air flow direction through channels.

FIG. 7. Cross-sectional view of channels showing air flow.

FIG. 8. Schematic of the cooling garment where the two layers are tacked together to form one big channel.

FIG. 9. Diagram of space between tacks in single channel embodiment of the cooling garment. Cross-sectional view between tacks (a) with porous filler material support (b) without any filler material.

FIG. 10. Water vapor flux equation.

FIG. 11. Mass transfer coefficient as a function of channel height.

FIG. 12. Test results using the cooling shirt on a sweating thermal manakin at National Personal Protective Technology Laboratory (NPPTL).

FIG. 13. Test results using the cooling shirt on a sweating thermal manakin at NC State, where the cooling shirt had non-optimized permeability.

### DETAILED DESCRIPTION OF THE INVENTION

Current methods for cooling are inadequate in hot, humid climates, especially for workers who must wear heavy layers of protective clothing while completing manual labor. The garment disclosed herein provides a lightweight, easy-to-manufacture, cooling mechanism which can be worn beneath heavy layers without risk of failure or crushing, and is effective even in the hottest and most humid climates.

Active air flow is critical for cooling mechanisms in hot, humid climates where sweating provides ineffective cooling due to the high ambient humidity. The present cooling garment utilizes a small fan to promote air flow through one or many air channels in the garment, which are supported by a porous filler material to prevent channel collapse and ineffective cooling. Preferably, the entire garment weight is less than 4 lbs, making the cooling garment a much lighter option than alternative methods of cooling such as ice packs and liquid cooling systems. The garment is made of flexible material with the fabric layer closest to the skin having high permeability, for maximal comfort and sweat-wicking capabilities. The permeability of the fabric layer closest to the skin may increase in various locations through the channel to strategically improve air flow distribution. The permeability of the outer layer of fabric is near impermeable.

In the summary of the invention above and in the Detailed Description of the Invention, and by the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, etc. are optionally present. For example, an article “comprising” (or “which comprises”) component A, B, and C can consist of (i.e. contain only) components A, B, and C, or can contain not only components A, B, and C but also one or more other components.

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending on the variable being defined). For example, “at most 4” means 4 or less than 4, and “at most 40%” means 40% or less than 40%. When, in this specification, a range is given as “(a first number) to (a second number)” or “(a first number)-(a second number)”, this means a range whose lower limit is the first number and whose upper limit is the second number. For example, “2 to 10 millimeters” means a range whose lower limit is 2 mm, and whose upper limit is 10 mm.

Garment means an item of clothing and may include a shirt, a vest, a collar, trousers, a pair of shorts, a hat, a sweat band, and the like.

Moisture-wicking under layer means a layer of fabric on the inside of the garment, making contact with the wearer's skin and having the ability to pull moisture away from the skin. Non-limiting examples of materials suitable for the layer include Supplex®, Meryl®, Dual Fit Strong® (MITI, Italy), IsoChill (Under Armour), and Coolmax® (DuPont—now Invista).

Outer layer means a second layer of fabric overlaying the moisture-wicking under layer, where air channels of various types can be formed between the two layers, and the outside of the layer is exposed to the outside environment or additional clothing items a wearer may place over top of the garment.

Air permeability means a measure of the flow rate of air per unit area at a given differential pressure, generally a measurement of how easily air is passed through fabric and indicating a fabric's breathability. Higher air permeability indicates higher passage of air through the fabric, and thus higher breathability. Lower air permeability traps air inside a space confined by the low permeability fabric or material.

Channel means a high aspect-ratio passage which air or gas can pass through. Channels in the present invention are contained between the wicking layer and the outer layer.

Porous filler material means a material that allows air to freely flow through it, and is a conformal lattice with low pressure drop. It must be deformable enough to move with the shirt, but sufficiently stiff to prevent channel collapse, all while having an open structure to allow air flow with low pressure drop. It should have mechanical shape memory, so if it is crushed with enough force it will return to its original shape when the load is removed. The porous filler material may be 3D mesh, 2D mesh materials folded onto themselves, open cell foam, open foam material, hog hair air filters, open cell rubbery meshes, open cell structured metamaterials, lattice-designed photopolymers, spacer fabrics or equivalent materials that fit the necessary specifications of flexibility, structure, and air flow.

Above ambient pressure gas supply means a device capable of providing a gas at a pressure higher than the ambient pressure outside the garment. The device can generate higher than ambient pressure from ambient air, or it may have pressurized gas in a container. Non-limiting examples of devices that generate higher than ambient pressure gas may include a pump, a fan, a positive displacement pump, a blower, and the like. Non-limiting examples of devices that have pre-pressurized gas in a container may include a compressed gas tank or a compressed breathing air tank and the like. Another example of an ambient pressure gas supply is air expelled by a person's mouth or nose. The gas may be ambient air, ambient air that has been conditioned to alter the temperature or humidity or a compressed gas: non-limiting examples include CO₂, N₂, Ar, O₂, mixtures thereof, and the like. The word “supply” in the phrase “above ambient pressure gas supply” is a noun.

Inlet means an air connection opening or fitting between the above ambient pressure gas supply and a first end of an air channel, where the connection provides a sealed entrance for air to be directed in and through the garment without any escaping into the outside environment. This can be a hose barb, a threaded fitting, an orifice, a funnel, or the like.

Water-wicking capability means a measurement of how quickly a fabric can move a liquid upward through the fabric, through capillary action. Moisture-wicking fabrics have many tiny capillaries which act to pull moisture away from a liquid source, for example the skin of a wearer of a moisture-wicking garment. The velocity of fabrics' liquid uptake can be measured using DIN 53924. Dipping standardized sizes of swatches of moisture-wicking fabrics in standardized volumes of water provide measurements of water-wicking capability that allow different fabrics to be compared to one another by their suction speed against water. Higher water rise relates to a higher water-wicking capability.

Outlet means an air connection opening or leak between a second opening in a channel and the outside environment. The outlet should be positioned to allow for the longest path of air movement within the channel or channels after introduction to the channels by the inlet. The outlet may direct air back toward the above ambient pressure gas supply generator to recycle air through the garment, or it may allow warmed air to escape through a direct opening or area of increased permeability at the end of the channel.

Tack means a connecting mechanism between the outer fabric layer and inner fabric layer that occurs only in a point location. The tack may be achieved through any sewing a stitch known to one in the art, nonlimiting examples including a “French Tack” or a “tie tacking stitch.” The tack may also be achieved through welding, strong fabric glue, fusing, small solid plastic or metal connectors, or other similar methods of holding two layers of fabric together.

Ballooning means the effect of an outer fabric layer bulging out and away from its initial position near an inner fabric layer due to air flowing between the two fabrics pushing out against the second layer. Ballooning is more likely to occur when the outer fabric layer is more impermeable, as any air pushing against the layer will have no means of escaping through the fabric. It is also more likely to occur when there is a larger distance between attachment means of the two layers, whether that be sides of channels or tacks holding the layers together in specific locations, because there is more space for the fabric to expand due to its inherently flexible nature. Ballooning reduces heat transfer because it increases the hydraulic diameter of the channel, decreasing the mass transfer coefficient and reducing sweat evaporation. Ballooning is non-desirable since it reduces sweat evaporation efficiency.

Hydraulic diameter is a term understood by A Person Having Ordinary Skill in the Art and is a commonly used term when handling flow in noncircular tubes and channels. Hydraulic diameter is further defined in Fundamentals of Heat and Mass Transfer by Theodore L. Bergman, Adrienne S. Lavine, Frank P. Incropera, David P. DeWitt, April 2011, which is incorporated by reference herein.

Overheating is a serious problem, especially in hot, humid, climates, and especially for workers in these climates that require many hot, heavy layers of protective gear while they complete arduous tasks. The present cooling garment overcomes limitations in the prior art to provide a lightweight cooling mechanism, effective in hot, humid climates where sweating is ineffective. The garment is specifically designed to promote the most effective airflow for cooling, with self-regulating capabilities to prevent overcooling. The cooling channels of the garment are sturdy for being worn underneath heavy protective gear without loss of cooling ability, while the garment itself remains flexible enough to provide comfort for the wearer. The garment is small, portable, and lightweight so it can be carried by a user with ease until being worn with maximal comfort.

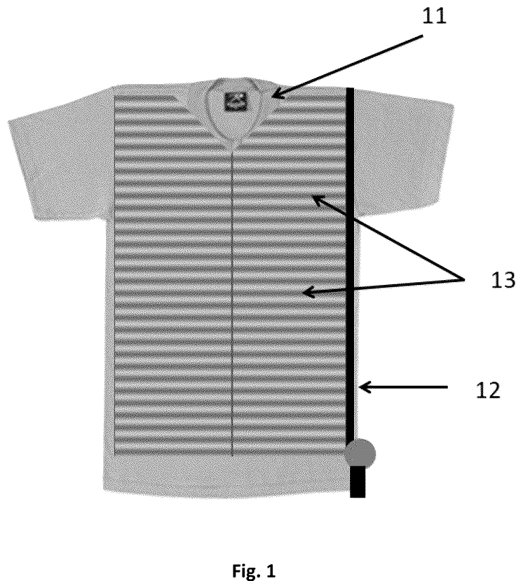

The garment works in both hot-humid and hot-dry climates, and uses the body's natural production of sweat to regulate the amount of cooling to a wearer. This prevents over-cooling, which is a serious risk with other cooling methods like ice packs. The garment wicks sweat from a wearer's body with an under-layer of fabric made from a fabric with moisture-wicking capabilities. A second layer of near impermeable fabric connects to the outside of the moisture-wicking layer and forms a single channel or an array of channels between the layers. Air is then forced through the channels, which have a small hydraulic diameter to improve the mass transfer coefficient, evaporating the sweat and producing a cooling effect. A small fan may be attached to an inlet to the channel(s) to blow air through the specially designed garment. In one embodiment, the garment is worn over the torso and air blows from a fan worn on a belt (FIG. 1). Air flows upward through the garment in a non-limiting example and evaporates sweat from the under layer of the garment as it flows across the torso of the wearer. In an example, the air and evaporated sweat are vented out the far side of the shirt, but other configurations of air flow directions and outlets are possible. Other possibilities include, but are not limited to channels aligned in horizontal, vertical, diagonal, and curved with upward, downward, and side-to-side airflow.

The importance of small hydraulic diameters is emphasized in the design of the cooling garment. Even in very humid climates, it is still technically possible to evaporate enough sweat to keep up with the rate of metabolic heat generation because ambient air is almost never saturated with water (typical relative humidity levels along the U.S. Gulf Coast are 40-80%, not 100%). The problem in humid climates is that the rate of evaporation of the sweat is not fast enough to effectively cool a person due to the body's low surface area and the low driving force for mass transfer. Smaller hydraulic diameters increase the rate of mass transfer for evaporating water from the liquid phase (sweat) to the vapor phase (air). By idealizing the hydraulic diameter present in the air channel(s), determined by channel height, improved cooling effects can be achieved. In other words, the rate of sweat evaporation can be increased to a maximum that is able to keep up with the rate of sweat production, and produce cooling, even in very humid climates.

The garment works by blowing air through one or many channels in the cooling garment, which may be a shirt, vest, or equivalent garment. The design specifications for producing the most effective cooling, described herein, may be translated to other wearable garments and underlayers as well. The evaporative process takes place within the channel(s) of the garment and results in cooling which is dependent on the body's rate of metabolic heat production. It is necessary to supplement the body with active air flow to prevent a metabolic heat load from building up in absence of sufficient air flow for evaporation.

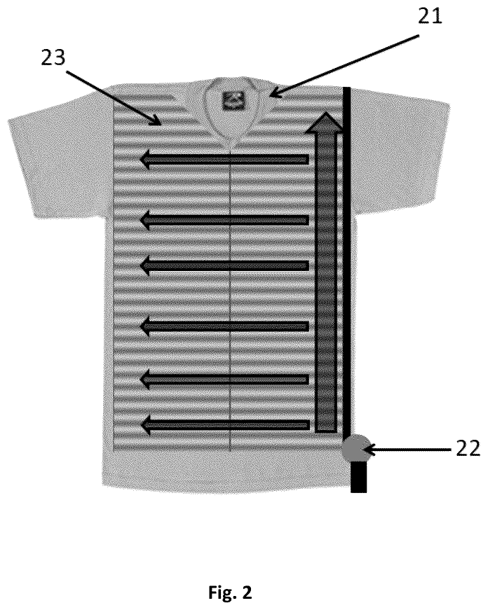

FIG. 1 shows a schematic of one method of incorporating channels with small hydraulic diameter into the moisture wicking garment, including sweat wicking garment layer (11), inlet manifold front and back (12), and horizontal cooling channels that have small hydraulic diameters attached to the sweat-wicking layer (13). FIG. 2 shows a schematic of air channels with an example of the flow direction, including sweat-wicking fabric undergarment (21), air inlet from small fan—a vertical fabric manifold (22) and cooling channels (23) created by stitching an air impermeable layer onto the wicking t-shirt (potentially with a 3D mesh fabric between them to act as a spacer). Arrows show the direction of air flow across the torso and out the other side of the shirt, providing evaporative cooling.

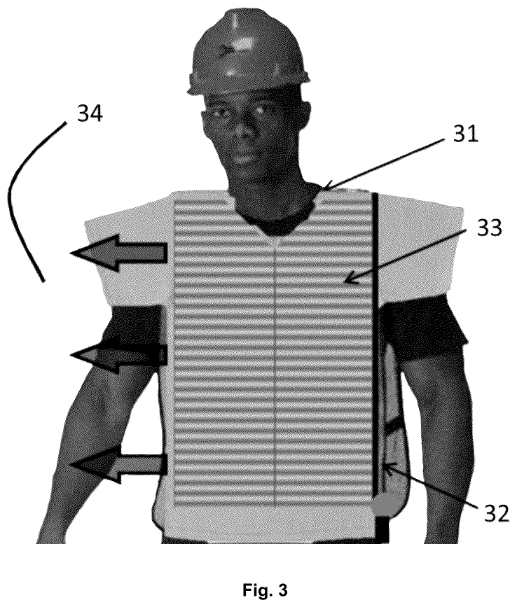

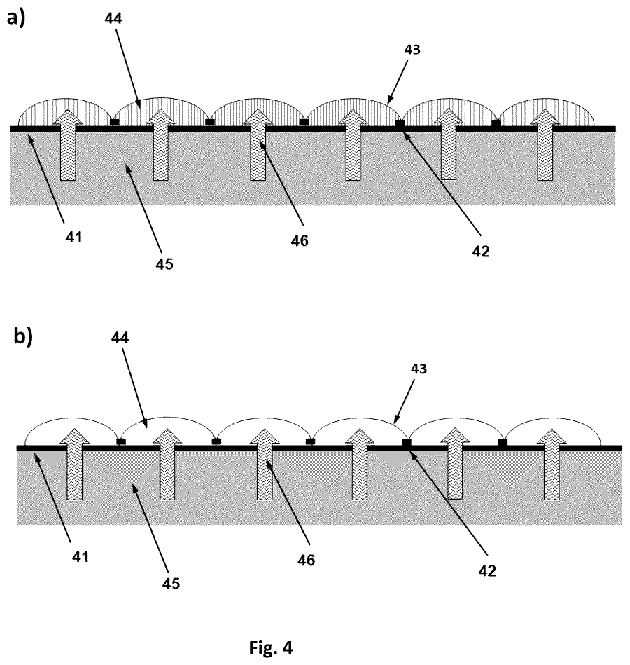

The cooling garment may have a small fan for inducing air flow through a specially designed garment worn over the torso. The garment is made from an under layer of moisture-wicking fabric, and a near-impermeable outer layer attached to the under layer, the layers forming a channel or channels with a small hydraulic diameter (FIG. 1 and FIG. 4) so moisture can be wicked and distributed into the channel(s) where active air flow promotes evaporation. The evaporated moisture is vented toward channel outlets, which can be arranged in various configurations. FIG. 3 shows the cooling garment in use, including a close fitting sweat-wicking under layer (31), an inlet manifold front and back (32), vertical or horizontal cooling channels attached to the garment (33), and arrows showing direction of air flowing out (34).

The cooling garment uses a lightweight, portable power source such as a battery (nonlimiting examples include rechargeable lithium batteries, NiMH batteries, and others). For example, a Li-ion batteries used in a lightweight power tool battery pack that lasts 4 hours weighs about 0.4 lb (˜180 g). The entire cooling garment may weigh less than about 4 lb. Compared to alternative liquid cooling systems and ice packs, the battery-powered fan provides a much lighter-weight option to achieve cooling. For example, removing 250-400 W (850-1400 Btu/hr) of metabolic heat (moderate to hard work) requires melting 6-10 lb of ice per hour. To remove the same amount of metabolic heat, wearer of the garment produces 0.85-1.4 lb of sweat, and this is produced from drinking water that the wearer would have to drink regardless of cooling mechanism. The light weight and simplicity of the garment provides safety, ease, and comfort to those working in hot, humid climates.

FIG. 3 shows how the cooling garment can be worn by first responders, construction workers and other personnel. The moisture wicking shirt with attached channels supported by a 3D mesh fabric (other embodiments of this invention include a single large channel formed by tacking an air impermeable outer layer to a wicking bottom layer in various locations with 3D mesh as a support) is worn next to the skin and the fan and batteries are worn on a belt, for example. The air flow runs from the fan to a manifold up the side of the garment, air flows sideways through channels in the shirt and exits through an outlet or outlets on the opposite side of the body. A cross section of the channels and how they are attached to the wicking fabric is shown in FIG. 4, which illustrates a non-limiting example using wide channels produced by sewing an outer air impermeable layer to the bottom wicking layer with a porous filler material layer in between. For example, FIG. 4 shows a drawing of how channels are produced by sewing an air impermeable layer (43) to the moisture wicking inner layer (41) of the garment with seams (42), and with 3D mesh forming airflow passages (44) that have a hydraulic diameter of about 0.2 inches and used to cool the adjacent skin (45) by transporting water from sweat (46).

Many synthetic “technical” fabrics exist with moisture-wicking capabilities, containing many small capillaries that bring moisture from the skin to the outer layer of the fabric where the moisture can be evaporated. Fabrics with this characteristic can be used as the moisture-wicking under layer in the cooling garment, which is worn closest to the skin of the wearer. They may be strong, durable, comfortable, and have excellent moisture wicking properties. Non-limiting examples include Supplex®, Meryl®, Dual Fit Strong® (MITI, Italy), IsoChill (Under Armour), and Coolmax® (DuPont—now Invista). Supplex® is a synthetic stretchable fabric with both air and water permeability. Meryl® is an ultralight performance polyamide fabric with high breathability, high wicking, and quick drying capabilities. Dual Fit Strong® is a highly durable, high elasticity, high moisture-wicking fabric with a particular weaving that reduces contact points between the worn garment and the skin. IsoChill is a nylon knit fabric that disperses body heat, making it feel cool to the touch. Coolmax® (DuPont—now Invista) is a quick-drying fabric with proprietary four channel polyester fibers that are weaved together in cross sections to allow air to flow through the fabric.

Importantly, the wicking layer should have a water wicking capability (measured using DIN 53924) to produce a water rise of at least 1.5 cm measured at 60 s of exposing the bottom 5 mm of a swatch of fabric (6 inches tall×2 inches wide) into a beaker of water. The wicking effectiveness of the fabric could produce a water rise anywhere from 1 cm-9 cm measured at 60 s of exposing the bottom 5 mm of a swatch of fabric into a beaker of water. A more preferred embodiment has a water rise of 3 cm-8 cm. The most preferred embodiment has a water rise of 6 cm-7.5 cm.

Channels for air flow are produced by attaching a near-impermeable outer layer of fabric to the moisture-wicking under layer of fabric. The outer layer and formed channels must be flexible enough to not interfere with the wearer's movements. With this flexibility comes the risk of channel(s) collapsing. One solution is to have a porous filler material between the inner wicking layer and the outer air impermeable layer to provide room for air to flow and prevent channel collapse. The filler material may be a 3D mesh fabric, or any other similar material that is a conformal lattice with low pressure drop, is stiff enough to prevent channel collapse while still being flexible to not inhibit movement, and will return to its original shape upon heavy crushing.

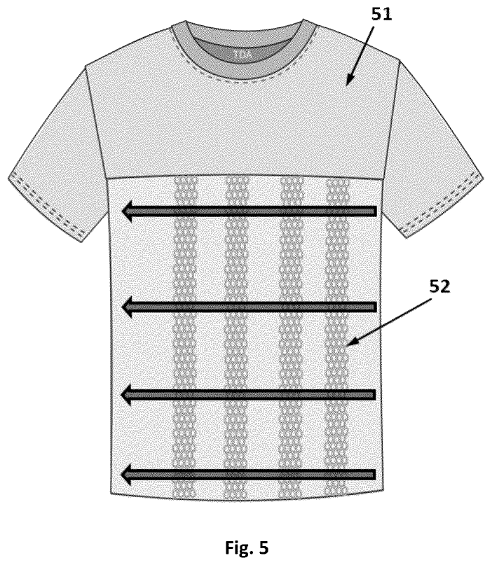

The effective mass transfer coefficient, which drives sweat evaporation, is dependent on the hydraulic diameter of the channels. The hydraulic diameter for air flowing through parallel plates is typically calculated as twice the distance between the plates. However, in this application, only the bottom layer is wetted with water as shown in FIG. 4, increasing the hydraulic diameter to four times the distance between the wicking and air impermeable layers. This means that thinner channels leads to better mass transfer. However, a balance must be struck between a high mass transfer coefficient and excessively thin channels leading to high back pressure (limiting air flow). One example solution is to use very thin porous filler material (for high mass transfer), but only have it in certain locations to prevent channel collapse, instead of having it everywhere (to reduce pressure drop) such as in FIG. 5 (showing the location of the 3D mesh strips without the air impermeable upper layer in view). Strips of 3D mesh (52) are spaced out atop the moisture-wicking under layer (51), and arrows show how air flows in channels across the strips of 3D mesh.

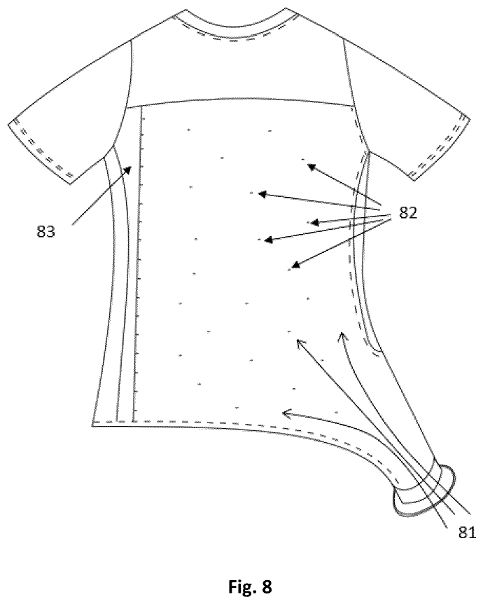

Due to the large air flow area, even if some of the area is cut off (i.e., channel collapse), there is still considerable air flow and significant cooling. In addition, intermediate manifolds can be added to reroute air around obstructed channels. Alternatively, if the shirt is constructed with one big channel (where the two layers are tacked together) such as FIG. 8, then air can easily reroute around obstructions. Also, there is clearly a tradeoff between comfort and the intended use. For example, if the cooling garment is to be worn either as an outer garment or under a light shirt there is little reason to worry about channels crushing. In this case, thinner and more flexible porous filler material can be used in areas which maximize comfort. However, if it is to be worn under heavy equipment, then crush resistance is important. In this case, thicker 3D mesh that is more evenly distributed could be used to prevent channel collapse.

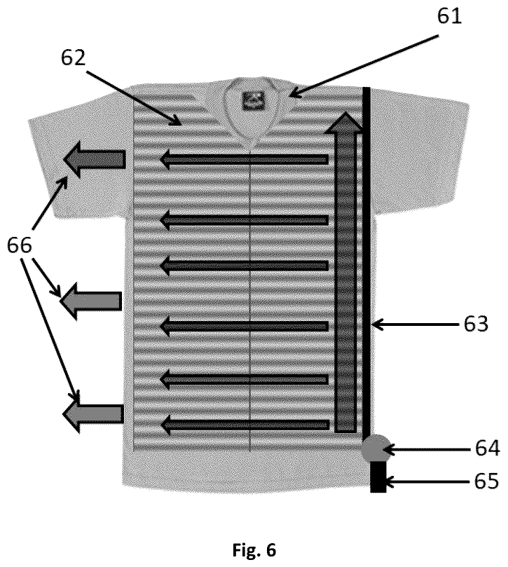

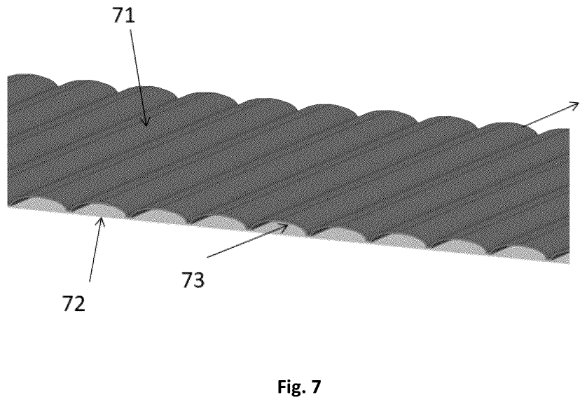

Sweating is the basis of how the cooling garment works. The amount of heat removed by the cooling garment is dependent on the amount of sweat generated, and thus is regulated by what the body needs at a given moment. Channel(s) with a small hydraulic diameter are incorporated into the shirt, and air is forced through them with a small battery powered fan (FIG. 6). FIG. 6 shows a schematic of a cooling garment, including a snug fitting, sweat-wicking fabric as the base layer (61) to which the air flow channel(s) (62) are attached, an air manifold (63) connected to a fan (64) powered by a battery (65), and air flow across the torso and out the other side (66). The small hydraulic diameter of the channel(s) increases the rate of sweat evaporation. By increasing the rate of sweat evaporation, the garment is designed to keep up with the body's metabolic rate of heat generation and reject all of the heat produced even when working very hard and in very humid, hot climates. A schematic representation of a cross sectional view of how these channels are formed in the cooling shirt is shown in FIG. 4 and FIG. 7. The shirt consists of at least two layers, forming channel(s) between them. The under layer, closer to the skin, is composed of moisture-wicking fabric so sweat is transported from the skin and to the outer surface of the wicking fabric. An air impermeable layer is stitched, or otherwise attached, to the under layer to form the channel(s). By using an air impermeable fabric as the outer layer of the channel, air flow is promoted across the garment through the openings in the channel(s) since it cannot leak through the outer fabric and into the environment.

While it may not be possible to have the outer layer be 100% air impermeable, it must have an air permeability less than 200 L/m²/s when subjected to 125 Pa of air pressure. It is important that the outer layer be near-impermeable so that air within the channels cannot flow through the outer layer and be released into the environment without providing cooling. The permeability of the wicking fabric is also important since air can leak out through it as well. The wicking layer cannot be fully air impermeable, or else it will also be water impermeable. If it's only slightly permeable to air, then the air that does leak has nowhere to go and can cause the wicking layer to “balloon” away from the skin and reduce heat transfer, so there is some benefit to having a more permeable wicking layer. In an embodiment, the permeability changes from inlet to outlet, starting with low permeability and ending with highly permeable mesh. This encourages air to flow across the chest instead of all coming out at the inlet. The permeability of the wicking layer should be in the range of 100-10,000 L/m²/s when subjected to 125 Pa of air pressure. In a preferred embodiment, the pressure drop is 250-5000 L/m²/s when subjected to 125 Pa of air pressure. In the most preferred embodiment, the permeability is 500-2000 L/m²/s when subjected to 125 Pa of air pressure. FIG. 4 shows a diagram of how fabric layers are attached to form air channels used for evaporative cooling (cross-section of cooling shirt/vest). The moisture wicking fabric (41) is attached to the air impermeable layer (43) with high resistance to air leakage out by attachment points formed from stitching (42) wherein the distance of the rows of stitching is mostly unimportant, forming air flow passages supported by 3D mesh (44) to cool skin (45). Water from sweat (46) is wicked by inner fabric and evaporates in the air passage, thus cooling the wearer. The height of these channels is extremely important since it determines the hydraulic diameter, and hence the mass transfer coefficient. The height can be controlled by inserting a porous filler material between the wicking and air insulating layers. FIG. 4b shows the same features as FIG. 4a, but in a section where there isn't the strip of 3D mesh. FIG. 7 shows a plurality of channels made from non-breathable fabric outer layer (71) and a sweat wicking fabric (72) to be used next to skin. The assembly forms air channels (73).

While the height of the channels is important (since that affects the hydraulic diameter and the mass transfer coefficient), the width of the channels is much less important. However, channel width should still be within 0.25 inches to 4 inches in the garment with many channels. If it's too low, then we introduce too much back pressure (due to the no-slip condition at the channel walls) and restrict air flow. If it's too wide, then there is space for the upper layer to balloon away from the wicking layer, reducing the channel height and the mass transfer coefficient. In a preferred embodiment of the cooling shirt design, the channels should be 0.5 inches to 2 inches wide. In the most preferred embodiment, the channel widths are 0.75 inches to 1.5 inches. Depending on the size of the intended wearer and the width of the channels, the garment may have 30-50 channels.

One embodiment of the invention is a shirt where the two layers (the inner wicking layer and the outer air impermeable layer) are tacked together without any channels sewn in. This means that the whole area between the two layers is one big channel. This way, there is no need for redistribution manifolds and air can easily reroute around an obstacle. The important parameter that generates a large mass transfer coefficient is the hydraulic diameter. The hydraulic diameter is dependent on the height of the channel, the distance between the two layers. So, even with only a single channel, as long as the distance between the layers is kept small (by having the tacks at appropriate distances to prevent the layers from spreading too far apart), then the hydraulic diameter will still be small and the mass transfer coefficient will be large, leading to efficient sweat evaporation. Only the bottom wicking layer is part of the wetted perimeter (regarding water). This is much less than the typical definition of the wetted perimeter (regarding air) causing the hydraulic diameter to be four times the distance between the layers instead of twice that distance.

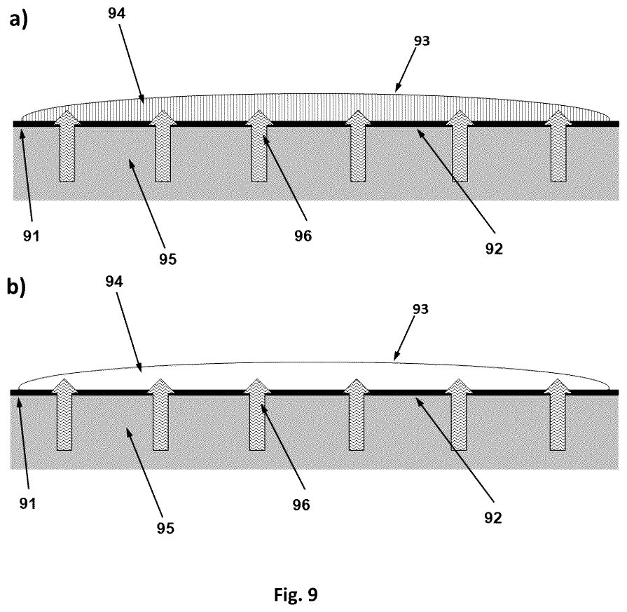

FIG. 8 shows a diagram of a cooling shirt where the two layers are tacked together to form one big channel. Air enters through the air inlet (81), and tacks (82) stitch the two layers together with a small strip of very thin 3D mesh (83) to increase pressure drop and improve air flow distribution. This arrangement of tacks prevents the two layers from separating by more than a few mm in any location, ensuring a small hydraulic diameter, a high mass transfer coefficient, and efficient sweat evaporation. This scenario is essentially a pair of infinite parallel plates, where the hydraulic diameter is just four times the distance between the plates (i.e. the distance between the layers). FIG. 9 shows the single tacked area in detail. Tacks (91) hold the outer air impermeable layer (93) and the bottom wicking layer (92) together, forming an aid channel (94) supported by 3D mesh. The wearer's skin (95) exudes sweat, and the water from sweat (96) is transported into the channel. FIG. 9b shows the same features as FIG. 9a, but in a section without 3D mesh.

The cooling garment is essentially a more efficient fan. It is wearable, non-bulky, and provides relief from heat like standing in front of a fan on a hot day, except it is more thorough in cooling the entire body. A small fan is placed at the inlet of the channel(s) in the garment, and forces air rapidly over the skin (i.e. forced convection). Importantly, the heat and mass transfer coefficients are much higher for flow in a channel with a small hydraulic diameter than flow over a large surface, and these coefficients determine the rate of sweat evaporation. The inverse relationship between hydraulic diameter and heat and mass transfer coefficients means that small channels will improve sweat evaporation. By idealizing the dimensions of the channels, the garment can take advantage of the body's natural cooling system even in humid environments. An advantage of relying on the body's cooling system is that it essentially eliminates the risk of over-cooling. When the body is sufficiently cooled with the assistance of the garment, it will stop producing sweat, causing additional cooling to cease. Once the body gets hot again, sweating will resume along with evaporative cooling assisted by the garment.

The channel(s) in the cooling garment have small hydraulic diameters so the mass transfer coefficient for evaporating water is large enough for sweat evaporation to keep up with sweat generation. An under layer of fabric with moisture-wicking capabilities is worn next to the skin, so sweat is transported into the channel formed between the under layer and an outer, impermeable layer of fabric. The outer layer must be impermeable, or near-impermeable, so that air flows through the channel and promotes evaporation rather than leaking out of the channel through the outer fabric. Inside the channel, the rate of evaporation is high, and it is important to keep that closed off from a humid outer environment which would cause the channels to lose their efficient evaporative capabilities. The two layers of fabric may be joined by knitting, RF welding, heat welding, stitching, gluing, tacking, and other equivalent methods. The walls of the channels must be flexible so that a user can move freely and not be constricted. The channels are supported by a porous filler material to eliminate the risk of channel collapse, yet they may still be attached to a fabric air manifold so if extremely heavy weigh pinches a portion of the garment closed, air will be rerouted. A variety of manifold designs can deal with the related issues of increasing heat/mass transfer rates, increasing flow rate, decreasing pressure drop, and preventing/mitigating the effects of channels being crushed or pinches off.

In one example the garment is a shirt designed so that the rate of sweat evaporation is increased to the point where the body can reject all of the heat it generates even during hard work in hot-humid climates. This is done by incorporating one or more air flow channels with a small hydraulic diameter, and forcing air through it/them with a small battery powered fan at a total flow rate of about 10-40 ft³/min with an ideal flow rate of approximately 25 ft³/min.

Ideally, the cooling shirt generates as little pressure drop as possible because higher pressure drop requires a fan that consumes more power to generate the same air flow. Higher power consumption shortens the battery life or increases battery weight.

The ideal fan is small, lightweight, consumes a small amount of power, and produces a high airflow (considering the pressure drop of the cooling shirt). The garment may have additional functional features, such as zippers for easy removal, clips for attaching to other clothing items, optional openings to the environment, and other similar features.

The airflow needed to cool the wearer can be estimated at a reference condition of 88° F. and 55% RH by looking at the change in absolute humidity (kg of water/kg of dry air). The enthalpy at the reference condition is ˜68.7 kJ/kg of dry air. To simplify analysis, one can imagine the heat and mass transfer in two distinct steps (though they occur simultaneously in real life). First, isenthalpic water evaporation. The air flowing through the channels will saturate to its wet bulb temperature (when RH is 100%) at about 73.4° F. This accounts for approximately 4.3 grams of water evaporation per cubic meter of air. However, the wearer's body is considerably warmer than 73.4° F. This warms the air up, increasing its capacity to evaporate additional water. Since the small channels that improve mass transfer coefficient also improve the heat transfer coefficient, heat from the wearer goes into the air, warming it up and increasing its capacity to evaporate additional water. With a very high mass transfer coefficient and sufficiently long channels, the outlet air temperature will be approximately 95° F. at 100% RH. With a lower mass transfer coefficient or short channels, the outlet air will be cooler and/or less humid. Assuming outlet conditions of 95° F. and 100% RH, the cooling garment will evaporate approximately 27.3 grams of water per meter cubed of flowing air. To dissipate 500 W (a worker doing hard, physically demanding work), the garment needs airflow of ˜17 CFM. As the mass transfer coefficient drops, the outlet air temperature and RH will drop, reducing the amount of water that evaporates into the air.

The total amount of heat removed is given by equation 1,

$$Heat removed = Q = {\dot{m}}_{s w e a t} Δ {H}_{v a p} \qquad ( 1 )$$

where the rate of sweat evaporation (m) in lb/h multiplied by the heat of vaporization of water (ΔHᵥₐₚ≈1000 Btu/lb).

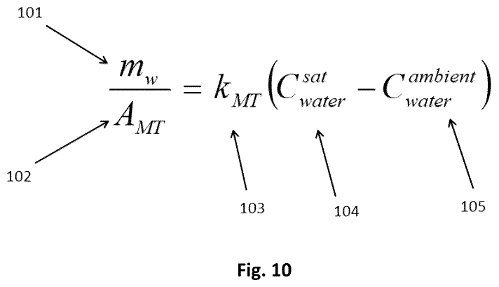

The rate of water (sweat) evaporation (m) depends on the mass transfer coefficient (k_{MT}) and the driving force for evaporation as shown FIG. 10. A_{MT} is the area for mass transfer, which in the case of the shirt is approximately the area of the torso and upper arms. The mass flux (\dot{m}/A_{MT}) can be expressed as a simple linear relationship in water vapor concentration difference because all the non-linear effects of the diffusion of water molecules in air, gas viscosity, and other fluid properties are lumped into the mass transfer coefficient. FIG. 10 shows the water vapor flux equation (kg/m²/h), the mass flux, or the mass flow rate (101) divided by the area for mass transfer (102) equals the mass transfer coefficient (103) times the difference of the concentration of water vapor next to the skin (104) minus the concentration of water vapor in the environment (105).

For evaporative cooling to work in high humidity (where the driving force for evaporation is low) the system must be designed so that the mass transfer coefficient for water evaporation is large. Mass transfer coefficient and hydraulic diameter are inversely related, so manipulating the hydraulic diameter of the channels to be smaller, the mass transfer coefficient becomes larger and the rate of sweat evaporation increases.

The cooling channel(s) in the garment are long across the chest (about 36 cm, 14 inches) and thin (about 4 mm high, 0.16 inch). Because the air flows through thin channels, the air is in laminar (streamline) flow. The mass transfer of water occurs when the sweat wicks into the garment's wicking layer, and then into the channel(s) containing the air flow (driven by the fan). To calculate the maximum rate of water (sweat) evaporation, the cooling garment is modeled as water evaporating from a channel made of infinite parallel plates into a laminar flow of air between the plates. This flow geometry is essentially a rectangular flat plate:

$$\frac{{k}_{M T} L}{D} = 0.3 \; 32 {( \frac{{Lv}^{0}}{v} )}^{1 / 3} {( \frac{v}{D} )}^{1 / 3} \qquad ( 2 )$$

The variables in equation 2 are: k_{MT} is the mass transfer coefficient, L is the hydraulic diameter of the channel(s), ν is the kinematic viscosity of air (40° C.), ν⁰ is the air velocity in the tubes, and D is the binary diffusion coefficient for water in air (D=0.277 cm²/see). The hydraulic diameter between parallel plates is typically 2 multiplied by the distance between the plates. However, since only the bottom surface (the wicking fabric near the skin) is wetted, in this application, the hydraulic diameter is calculated as 4 multiplied by the distance between the plates. For a given volumetric air flow rate, the gas velocity is inversely proportional to the cross-sectional flow area (the total length of the channels multiplied by the channel height, L/4. This means that the mass transfer coefficient is inversely proportional to L.

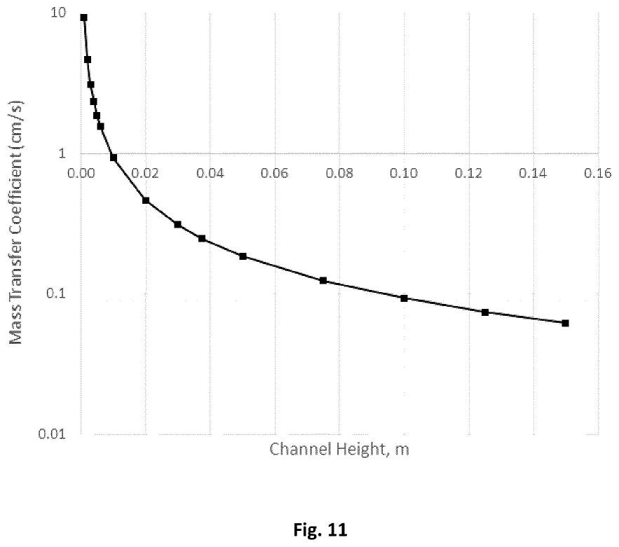

With a channel height of 2 mm, the air velocity is 5.24 m/s in the channel(s) (total air flow of 20 ft³/min), the mass transfer coefficient is k_{MT}=4.64 cm/s. At a channel height of 4 mm, gas velocity is approximately 1.75 m/s and the k_{MT}=2.32 cm/s. With a channel height of 10 cm, the gas velocity is 1.05 m/s and the mass transfer coefficient is 0.93 cm/s. FIG. 11 shows the mass transfer coefficient as a function of channel height.

A commonly used method for estimating the body surface area (BSA) is the Mosteller formula:

$$B S A ( {m}^{2} ) = {\{ ( Height ( cm ) ) ( Weight ( kg ) ) / 3600 \}}^{0.5} \qquad ( 3 )$$

A commonly used BSA for physiological calculations is 1.8 m², which using the Mosteller formula corresponds to an individual that weighs about 143 lb (65 kg) and is 5 ft 10 inches (1.8 m) tall. In general, the BSA breaks down approximately as: head=9%, chest=9%, abdomen=9%, upper back=9%, lower back=9%, arms (9% each), groin=1% and legs=18% each. Assuming that the shirt covers the front and back from chest to waist, the total surface area covered by the cooling garment is about 36% of the body's surface area, or 0.65 m² for a 1.8 m² person. However, due to the need for manifolds and easier manufacturing if channels are all straight lines, the cooling shirt can likely only cover ˜⅔ of the chest and back, reducing the effective cooling area to 24% of the body area or 0.43 m². The channels traverse the whole length across the front of the chest and the back (˜15″ long for a medium sized shirt) and covers substantially the full height of the shirt from chest to waist (˜18″ for a medium sized shirt). All calculations assumed of 0.43 m² of effective cooling area. Blowing air through thin channel(s) drastically increases the mass transfer coefficient (k_{MT}) inside of the channels, which increases the rate that water (as sweat) can evaporate from the shirt.

Example 1: 2 mm tall channels. A shirt that contains about 40 channels (20 on the front and 20 on the back) where the channels are 1.0-inch wide and 2 mm tall. The wetted perimeter is only the bottom edge of the channel. This garment removes about 980 W for a sweating wearer in a 90° F.-30% relative humidity environment (assuming that the wearer can sweat enough to keep up with the evaporation).

Example 2: 4 mm tall channels. A shirt that contains about 40 channels where the channels are 1.0-inch wide and 4 mm tall. The wetted perimeter is only the bottom edge of the channel. This garment removes about 489 W for a sweating wearer in a 90° F.—30% relative humidity environment (assuming that the wearer can sweat enough to keep up with the evaporation).

Example 3: 5 mm tall channels. A shirt that contains about 40 channels where the channels are 1.0-inch wide and 5 mm tall. The wetted perimeter is only the bottom edge of the channel. This garment removes about 390 W for a sweating wearer in a 90° F.—30% relative humidity environment.

Example 4: Example 1: 10 mm tall channels. A shirt that contains about 40 channels where the channels are 1.0-inch wide and 10 mm tall. The wetted perimeter is only the bottom edge of the channel. This garment removes about 195 W for a sweating wearer in a 90° F.—30% relative humidity environment.

Example 5: A tacked shirt with only one major channel. A shirt that contains one big open flow area (See FIG. 8) with tacks to restrict the flow area height. Cooling power will be nearly identical to a channeled shirt with a comparable distance between the wicking layer and the air impermeable layer. The benefit to a tacked construction is that air can more easily reroute around obstructions and there is reduced pressure drop (leading to more air flow). In fact, pressure drop can be so low that a strip of thin 3D mesh is needed near the exit to generate pressure drop to ensure even air flow distribution across the chest and back. The preferred embodiment of this design has tacks that are ˜2 inches apart to balance the competing interests of ease of manufacturing (fewer tacks are easier) with preventing the upper layer from ballooning away from the lower layer (more tacks will restrict ballooning) better.

Example 6: A tacked shirt similar to example 5, but with tacks spaced 1 inch apart. This reduces the maximum space between the two layers and the increases mass transfer coefficient.

Example 7: A tacked shirt similar to example 5, but with tacks spaced 0.25 inches apart. This is the minimum recommended spacing to prevent excessive pressure drop

Example 8: A tacked shirt similar to example 5, but with tacks spaced 4 inches apart. This is the maximum recommended spacing to prevent excessive ballooning of the outer layer away from the inner layer (reducing the mass transfer coefficient to an unacceptably low level).

Example 9: A shirt with air blown through it using a compressed air tank or an air compressor. In this embodiment, the compressed air can overcome nearly any back pressure that could be caused by a reasonably designed cooling shirt. In this embodiment, the back pressure of the shirt is not very important.

Example 10: A shirt with air blown through it using a fan/blower. In general, fans are capable of moving large quantities of air when subjected to little or no back pressure. Their ability to move air tends to drop off significantly when subjected to any considerable back pressure. So, when air is being blown through the shirt using a fan, for a given fan, there is an inherent tradeoff between pressure drop and volumetric air flow. In other words, higher pressure drop means reduced air flow for the a given fan. Alternatively, we can think of this based on the pressure drop. For a given pressure drop, there is a tradeoff between air flow and fan power consumption. Higher air flow requires a fan that consumes more power (leading to a bigger and heavier battery). In general, a lower pressure drop is preferred, since then we can achieve a higher flow rate with lower power consumption. In one embodiment, the pressure drop of the shirt should be between 0.25 inches of water (62.2 Pa), abbreviated as inWC, and 5 inWC. Preferably, the pressure drop is between 0.4 inWC and 2 inWC. Ideally, the pressure drop is 0.5 inWC to 1 inWC.

Example 11: A cooling garment with a battery powered fan. The higher the power consumption of the fan, the larger/heavier the battery powering it must be. Considering commercially available batteries in the year 2023, the fan power should be as low as possible. Ideally, the fan's power consumption is <40 W. In a more preferred embodiment, the fan's power consumption is <20 W. In the most preferred embodiment, the fan's power consumption is <10 W.

Example 12: Cooling garment used when Tdb=90° F. (32.2° C.) and dew point of 78.9° F. (26° C.), which gives Twb=81.5° F. and RH=70% (absolute humidity=0.0214 lb water/lb dry air). The garment operates for 4 hours on a single battery charge. The vest can cool an individual even during very heavy work, with 1263 Btu/hr (326 W) of metabolic heat. The garment weighs less than 4 pounds.

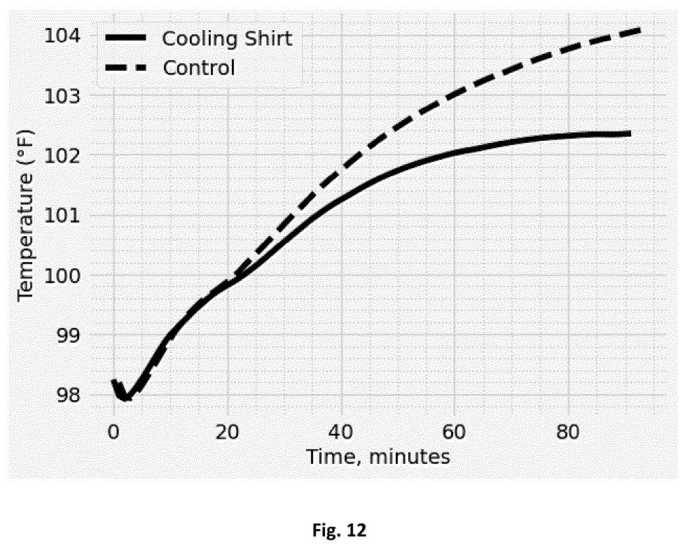

In April 2023, the cooling garments were tested with the National Personal Protective Technology Laboratory (NPPTL). NPPTL is a part of the National Institute of Occupational Safety and Health (NIOSH). NPPTL has the Newton thermal sweating manikin from Thermetrics as well as an environmental chamber to control the dry-bulb temperature (T_{db}) and relative humidity (RH). The Newton manikin has 30 independent zones measuring skin temperature and sweating rate. For the EOD cooling vest, the single most important parameter is the core body temperature. We also analyzed the skin temperature on the torso including the stomach, upper chest, lower back, and mid back. The manakin was dressed in a welding ensemble including a welding jacket, welding pants, winter gloves, socks, and a winter hat. All tests were performed with T_{db}=100° F. and RH=40% for 90 minutes. Additionally, the metabolic heat generation rate of the manikin was set to 5 METs, equivalent to moderate intensity exercise like walking at a 15-minute mile pace, kayaking, or doubles tennis. Multiple versions of the cooling shirt were tested and compared to tests where there was no cooling shirt (just a regular cotton T-shirt). The cooling shirt reduced the core body temperature after 90 minutes by up to 1.6° F. as shown in FIG. 12 (core body temperature at 90 minutes for the control setup was typically 104° F., whereas the cooling shirt reduced that to 102.4° F.). This may not seem like much, but since normal body temperature is 98.6° F. and heat stroke typically sets in around 104° F., there is only 5.4° F. that the core body temperature can rise before someone might suffer heat stroke (leading to hospitalization or death). A core body temperature reduction of 1.6° F. is nearly 30% of the space between normal core body temperature and heat stroke.

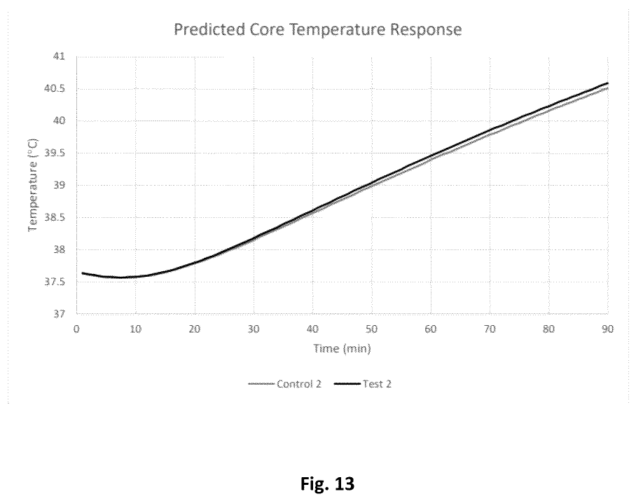

In March 2021, a cooling shirt was sent to NC State to perform the same tests using a sweating thermal manikin as were performed at NPPTL in April 2023. NC State used the same welding ensemble, the same sweating thermal manikin, the same 90-minute test time, and also set the metabolic heat load to 5 METs. The only difference was that instead of having T_{db}=100° F. and RH=40% (as was done at NPPTL), NC State set the ambient conditions to T_{db}=100° F. and RH=30%). In theory, this change should make the cooling shirt more effective (since it increases the driving force for sweat evaporation). However, the embodiment of the cooling garment at that time was not optimized for permeability in the wicking fabric or for air flow rate. As shown in FIG. 13, the cooling garment had only a tiny effect on the core body temperature. This clearly demonstrates that improperly designing the shirt can lead to very poor cooling performance. Because the margin between core body temperature and potential heat stroke is so small, it is crucial to design the cooling garment with specifically optimized measurements described herein. What may appear to be only a slight decrease in effectiveness by modifying the garment's specifications actually places a worker back in a state of risk.

What is claimed is:

1. A cooling shirt or vest garment, the cooling garment comprising:
a) A moisture-wicking under layer having an inner surface and an outer surface;
b) An outer layer attached to the outer surface of the moisture-wicking under layer, wherein the outer layer has an air permeability less than 200 L/m²s when subjected to 125 Pascals of air pressure;
c) At least one air channel between the moisture-wicking under layer and the outer layer, wherein the air channel has a channel width of 0.25-4 inches, and wherein the air channel is supported in at least some locations by a porous filler material with a height of 2-10 millimeters; and,
d) An above ambient pressure gas supply operably attached to an inlet of the channel to thereby cool the wearer of the cooling garment, wherein the gas supply forces air through the channel at a total flow rate of at least 10 and at most 40 cubic feet per minute.

2. The cooling garment of claim 1, wherein the channel width is 0.5-2 inches.

3. The cooling garment of claim 2, wherein the channel width is 0.75-1.5 inches.

4. The cooling garment of claim 1, further comprising 30-50 air channels.

5. The cooling garment of claim 4, wherein the 30-50 air channels have a width of 1 inch and a height of 2 millimeters.

6. The cooling garment of claim 1, wherein the moisture-wicking under layer is a fabric which has a water-wicking capability producing a water rise of 1-9 centimeters, measured at 60 seconds after exposing a bottom 5 millimeters of a swatch of the fabric into a beaker of water.

7. The cooling garment of claim 6, wherein the fabric has a water-wicking capability producing a water rise of 3-8 centimeters.

8. The cooling garment of claim 7, wherein the fabric has a water-wicking capability producing a water rise of 6-7.5 centimeters.

9. The cooling garment of claim 1, wherein the moisture-wicking under layer has a permeability which changes from the inlet of the channel to an outlet of the channel to encourage air flow across a wearer's torso.

10. The cooling garment of claim 9, wherein the permeability of the moisture-wicking under layer is 100-10,000 L/m²/s.

11. The cooling garment of claim 10, wherein the permeability of the moisture-wicking under layer is 250-5,000 L/m²/s.

12. The cooling garment of claim 11, wherein the permeability of the moisture-wicking under layer is between 500-2,000 L/m²/s.

13. The cooling garment of claim 1, wherein the total flow rate is 25 cubic feet per minute.

14. The cooling garment of claim 1, wherein the above ambient air pressure gas supply is either a compressed air tank, a blower, or a battery-powered fan.

15. A cooling shirt or vest garment, the cooling garment comprising:
a) A moisture-wicking under layer;
b) An outer layer attached to the outer surface of the moisture-wicking under layer, wherein the outer layer has an air permeability less than 200 L/m²/s when subjected to 125 Pascals of air pressure;
c) One large air channel between the moisture-wicking under layer and the outer layer, wherein the air channel is supported in at least some locations by a porous filler material with a height of 2-10 millimeters;
d) A plurality of tacks, wherein each tack connects the moisture-wicking under layer to the outer layer and prevents ballooning; and,
e) An above ambient pressure gas supply operably attached to an inlet to the channel to thereby cool the wearer of the cooling garment, wherein the gas supply forces air through the channel at a total flow rate of at least 10 and at most 40 cubic feet per minute.

16. The cooling garment of claim 15, wherein the tacks are spaced 0.25-4 inches apart.

17. The cooling garment of claim 16, wherein the tacks are spaced 2 inches apart.

18. The cooling garment of claim 16, wherein the tacks are spaced 1 inch apart.

19. The cooling garment of claim 15, wherein the moisture-wicking under layer is a fabric which has a water-wicking capability producing a water rise of 1-9 centimeters, measured at 60 seconds after exposing a bottom 5 millimeters of a swatch of the fabric into a beaker of water.

20. The cooling garment of claim 19, wherein the fabric has a water-wicking capability producing a water rise of 3-8 centimeters.

21. The cooling garment of claim 20, wherein the fabric has a water-wicking capability producing a water rise of 6-7.5 centimeters.

22. The cooling garment of claim 15, wherein the moisture-wicking under layer has a permeability which changes from the inlet to an outlet to encourage air flow across a wearer's torso.

23. The cooling garment of claim 22, wherein the permeability of the moisture-wicking under layer is 100-10,000 L/m²/s.

24. The cooling garment of claim 23, wherein the permeability of the moisture-wicking under layer is 250-5,000 L/m²/s.

25. The cooling garment of claim 24, wherein the permeability of the moisture-wicking under layer is between 500-2,000 L/m²/s.

26. The cooling garment of claim 15, wherein the total flow rate is 25 cubic feet per minute.

27. The cooling garment of claim 15, wherein the above ambient air pressure gas supply is either a compressed air tank, a blower, or a battery-powered fan.
